(19) United States
(12) Patent Application Publication
(10) Pub. No.: US 2023/0326594 A1
(43) Pub. Date: Oct. 12, 2023

(54) METHOD FOR PROVIDING AND UPDATING TREATMENT RECOMMENDATIONS

(71) Applicant: SPRINGTIDE CHILD DEVELOPEMENT, INC., Trumbull, CT (US)

(72) Inventors: Yiftah FRECHTER, Irvington, NY (US); Ksenya GUSAK, Truckee, CA (US); Michael CAMERON, Los Angeles, CA (US)

(21) Appl. No.: 17/715,914

(22) Filed: Apr. 7, 2022

(51) Int. Cl. G16H 50/20; G16H 20/00

(52) U.S. Cl. CPC G16H 50/20; G16H 20/00

(57) ABSTRACT

An apparatus and method for providing treatment recommendations based on a holistic assessment including a set of decision trees is presented herein. The method may include receiving a first set of responses based on prompts within each decision tree of a set of decision trees, each decision tree of the set of decision trees corresponding to a different aspect of a human activity. The method may further include outputting the prompts within each decision tree of the set of decision trees, at least one prompt being outputted based on one or more responses in the first set of responses. The method may also include determining at least one treatment recommendation based on the first set of responses to the prompts outputted for the set of decision trees. The method may further include outputting the at least one treatment recommendation.

### BACKGROUND

### Technical Field

The present disclosure relates generally to the provision of therapeutic interventions for a range of disorders. In some aspects, the range of disorders may include autism spectrum disorders.

### Related Art

In practice, the provision of therapeutic interventions for multi-faceted disorders may include separate interventions for addressing different aspects of the disorder(s). Current data collection methodologies, in some aspects, include unstructured data consisting of, e.g., therapist notes and personal identification information for a patient. This unstructured data is generally not usable to derive any insights from large populations of patients using existing data analysis methods. In some aspects, therapy may be provided a relatively-inexperienced therapist that may not be able to integrate information from multiple different assessments to formulate a treatment plan (e.g., a set of recommended treatments and/or assessments). Accordingly, there is an expected benefit to providing a system that provides recommendations based on a holistic analysis of available information across multiple areas of human activity.

### SUMMARY

Example implementations described herein include an innovative method for providing treatment recommendations based on a holistic assessment including a set of decision trees. The method may include receiving a first set of responses based on prompts within each decision tree of a set of decision trees, each decision tree of the set of decision trees corresponding to a different aspect of a human activity. The method may further include outputting the prompts within each decision tree of the set of decision trees, at least one prompt being outputted based on one or more responses in the first set of responses. The method may also include determining at least one treatment recommendation based on the first set of responses to the prompts outputted for the set of decision trees. The method may further include outputting the at least one treatment recommendation.

Example implementations described herein include an innovative apparatus for providing treatment recommendations based on a holistic assessment including a set of decision trees. The apparatus may include a user interface configured to present prompts to a user and receive responses from the user. The apparatus may further include a memory and at least one processor coupled to the memory that, when executing a program stored in the memory, is configured to receive a first set of responses based on prompts within each decision tree of a set of decision trees, each decision tree of the set of decision trees corresponding to a different aspect of a human activity. The at least one processor may further be configured to output the prompts within each decision tree of the set of decision trees, at least one prompt being outputted based on one or more responses in the first set of responses. The at least one processor may also be configured to determine at least one treatment recommendation based on the first set of responses to the prompts outputted for the set of decision trees. The at least one processor may further be configured to output the at least one treatment recommendation.

Example implementations described herein include an innovative computer-readable medium storing computer executable code for providing treatment recommendations based on a holistic assessment including a set of decision trees. The computer executable code may include instructions for receiving a first set of responses based on prompts within each decision tree of a set of decision trees, each decision tree of the set of decision trees corresponding to a different aspect of a human activity. The computer executable code may further include instructions for outputting the prompts within each decision tree of the set of decision trees, at least one prompt being outputted based on one or more responses in the first set of responses. The computer executable code may also include instructions for determining at least one treatment recommendation based on the first set of responses to the prompts outputted for the set of decision trees. The computer executable code may further include instructions for outputting the at least one treatment recommendation.

In some aspects, the example implementations may provide improved speed and accuracy of treatment recommendations by relatively-inexperience practitioners (e.g., therapists) by providing guidance through a set of interactive decision trees and integrating the information collected to output at least one treatment recommendation based on a holistic assessment. Furthermore, as discussed in the detailed description below, some aspects provide anonymized data for multiple patients that would otherwise be unavailable for analysis (e.g., due to Health Insurance Portability and Accountability Act (HIPAA) requirements). The anonymized data, in some aspects, may be analyzed by algorithms or may be used to train networks (e.g., neural networks) to further improve the accuracy or efficacy of the treatment recommendation and/or to update the decision trees used to generate the treatment recommendations. The anonymized data may further provide a benefit over locally-stored HIPAA-compliant data by allowing larger data sets that lead to improved performance of machine training of neural networks or other machine-trained algorithms. The anonymized data may also be made available to third-party researchers to perform analysis using components of the system.

### BRIEF DESCRIPTION OF DRAWINGS

FIG. 1 is a diagram illustrating components of a system for providing treatment recommendations based on a holistic assessment including a set of decision trees.

FIG. 2 is a diagram illustrating a data collection and anonymization operation for decision tree response data received at a plurality of local providers in accordance with aspects of the disclosure.

FIG. 3 is a diagram illustrating the use of anonymized data in an anonymized data storage to generate insights and update a set of decision trees stored in decision tree storage in accordance with aspects of the disclosure.

FIG. 4 is a diagram illustrating the use of anonymized data in an anonymized data storage to generate insights and update a set of decision trees stored in decision tree storage in accordance with aspects of the disclosure.

FIG. 5 is a diagram illustrating a portion of a feeding decision tree, in accordance with some aspects of the disclosure.

FIG. 6 is a diagram illustrating a first example display that may be presented to a user resulting from traversing a decision tree in accordance with some aspects of the disclosure.

FIG. 7 is a diagram illustrating a second example display that may be presented to a user resulting from traversing a second decision tree in accordance with some aspects of the disclosure.

FIG. 8 is a flowchart for a method of providing treatment recommendations based on a holistic assessment including a set of decision trees.

FIG. 9 is a flowchart for a method of providing treatment recommendations based on a holistic assessment including a set of decision trees.

FIG. 10 is a flowchart for a method of optimizing at least one decision tree based on a set of responses in accordance with some aspects of the disclosure.

FIG. 11A is a flowchart for a method of updating at least a first decision tree based on a set of responses in accordance with some aspects of the disclosure.

FIG. 11B is a flowchart for a method of updating at least a first decision tree based on a set of responses in accordance with some aspects of the disclosure.

FIG. 12 illustrates an example computing environment with an example computer device suitable for use in some example implementations.

### DETAILED DESCRIPTION

The following detailed description provides details of the figures and example implementations of the present application. Reference numerals and descriptions of redundant elements between figures are omitted for clarity. Terms used throughout the description are provided as examples and are not intended to be limiting. For example, the use of the term “automatic” may involve fully automatic or semi-automatic implementations involving user or administrator control over certain aspects of the implementation, depending on the desired implementation of one of the ordinary skills in the art practicing implementations of the present application. Selection can be conducted by a user through a user interface or other input means, or can be implemented through a desired algorithm. Example implementations as described herein can be utilized either singularly or in combination and the functionality of the example implementations can be implemented through any means according to the desired implementations.

Example implementations described herein include an innovative method for providing treatment recommendations based on a holistic assessment including a set of decision trees. The method may include receiving a first set of responses based on prompts within each decision tree of a set of decision trees, each decision tree of the set of decision trees corresponding to a different aspect of a human activity. The method may further include outputting the prompts within each decision tree of the set of decision trees, at least one prompt being outputted based on one or more responses in the first set of responses. The method may also include determining at least one treatment recommendation based on the first set of responses to the prompts outputted for the set of decision trees. The method may further include outputting the at least one treatment recommendation.

In some aspects, example implementations described herein may further be used to perform analysis on collected data. For example, some aspects provide a method and apparatus for identifying correlations between different decision trees relating to different areas of human activity. The method and apparatus, in some aspects, may identify correlations between different components (e.g., prompts, assessment recommendations, treatment recommendations, etc.) of one or more the plurality of decision trees. In some aspects, the method and apparatus may update one or more decision tress based on the identified correlations. Some aspects may additionally track implemented treatments/interventions and outcomes over time and perform a machine learning operation to identify optimized treatment plans based one or more received sets of responses to a set of responses based on prompts within each decision tree that are associated with the implemented treatments/interventions and outcomes.

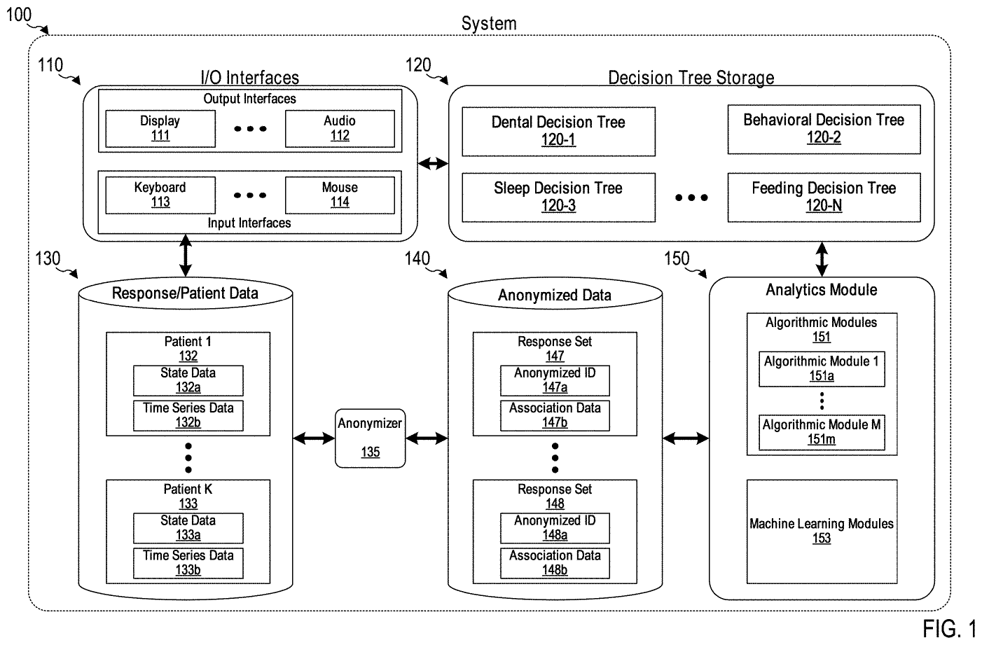

FIG. 1 is a diagram illustrating components of a system 100 for providing treatment recommendations based on a holistic assessment including a set of decision trees. The system 100 may include a set of input/output (I/O) interfaces 110 for interacting with one or more users. The I/O interfaces 110, in some aspects, may include a set of output interfaces including one or more displays 111, audio interfaces 112 (e.g., speakers) or other output interfaces for providing a set of prompts to a user. The prompts, in some aspects, may be obtained from a decision tree storage 120, e.g., from dental decision tree 120-1, a behavioral decision tree 120-2, a sleep decision tree 120-3, a feeding decision tree 120-N, or any other decision trees stored in the decision tree storage 120. In some aspects, the I/O interfaces 110 may also include a set of input interfaces such as a set of one or more keyboards 113, a mouse 114, a microphone (not shown), or other input interface devices.

The set of input interfaces, in some aspects, may receive responses to prompts output by the set of output devices based on one or more decision trees 120-1 to 120-N stored in the decision tree storage 120. The responses for a particular patient may be stored in a data structure associated with the particular patient, e.g., in patient data 132 or patient data 133 in a response/patient data storage 130. Each of patient data 132 or 133 may include data related to a current state 132a or 133a and time-series data 132b or 133b that tracks the state variables over time. In some aspects, the time-series data may also indicate an implemented intervention/treatment and a related outcome, the related outcome for a particular intervention may be identified/determined by a change in state variables after the treatment/intervention is implemented. Each of patient data 132 and 133 may, in some aspects, may include personal identifying information (e.g., patient name, address, etc.) for a patient as well as a non-personal identifier that may be used to identify related data sets without including protected patient information.

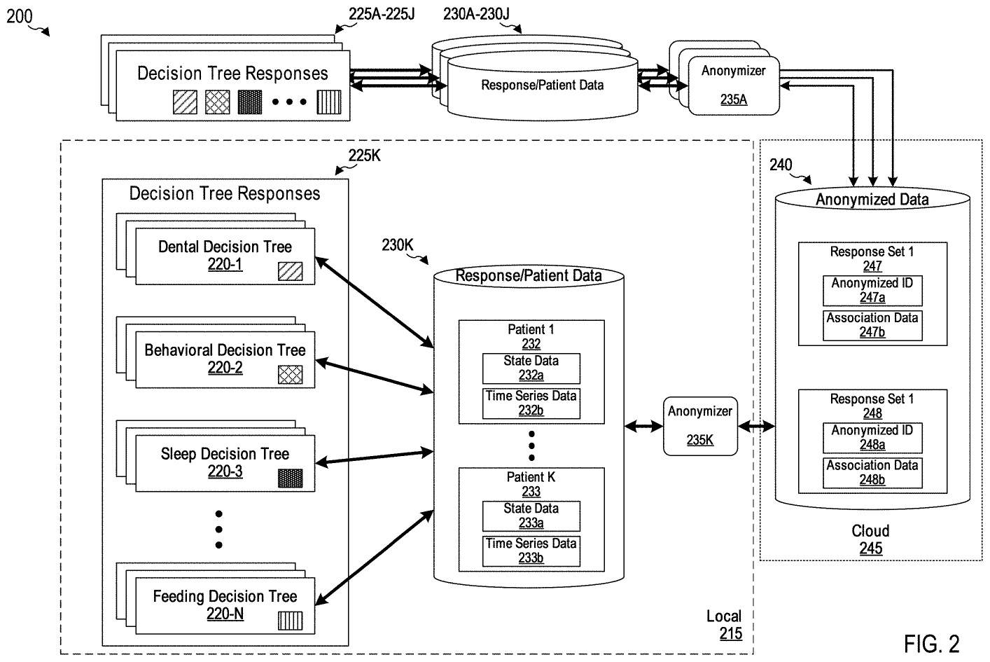

The response/patient data storage 130 may be processed by an anonymizer 135 to produce data for storage in anonymized data storage 140. The response/patient data 130 storage may be one of a local storage or cloud-based storage that is Health Insurance Portability and Accountability Act (HIPAA) compliant. The anonymized data storage 140 may be one of a HIPAA-compliant or a non-HIPAA compliant storage. The anonymized data storage 140 may include data structures (e.g., response set 147 and response set 148) identifying related sets of responses. Each response set (e.g., response set 147 and 148) may include an anonymized ID (e.g., anonymized ID 147a or anonymized ID 148a) that does not include patient-identifying information but that may be used within a HIPAA compliant system to identify a patient associated with the response set. Each response set (e.g., response set 147 and 148) may further include association data (e.g., association data 147b or association data 148b) regarding responses to prompts in a set of decision trees. In some aspects, the anonymizer 135 executes in the HIPAA-compliant environment before sending anonymized data (e.g., data that is not protected by HIPAA) to a non-HIPAA-compliant storage. In some aspects, the anonymized data storage 140 may receive anonymized data from multiple response/patient data storages associated with multiple providers using the decision trees 120-1 to 120-N as illustrated in FIG. 2.

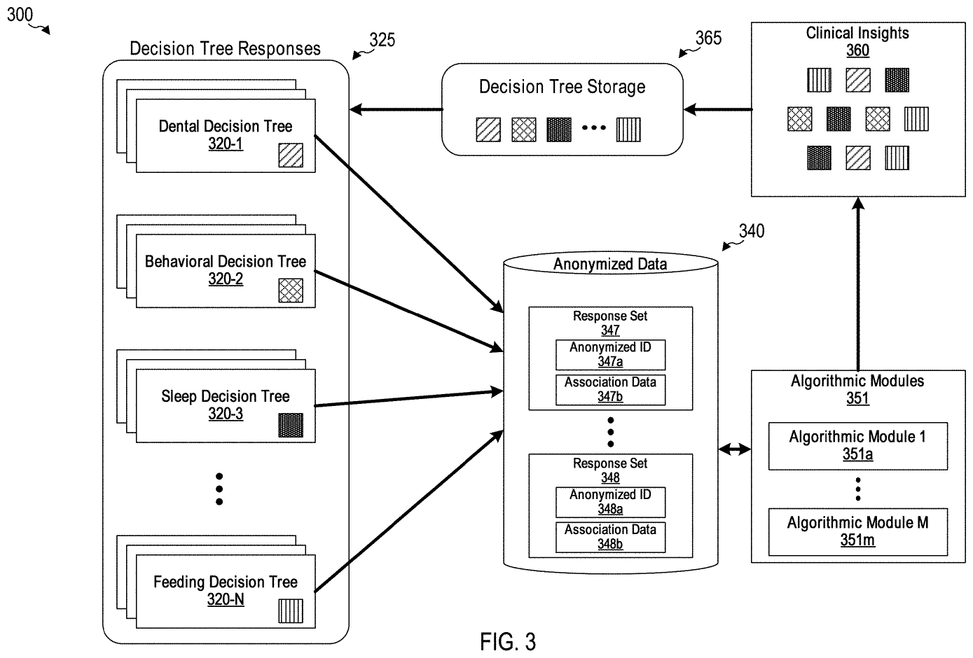

The system 100 may further include a set of analytics modules 150 that may access the anonymized data in anonymized data storage 140. The analytics module 150, in some aspects, may be implemented as a local module, a centralized module (e.g., in a cloud), or a combination of local and centralized modules (e.g., local clients that interact with a cloud-based analytics module and cloud-based anonymized data storage 140. In some aspects, a set of algorithmic modules 151 including algorithmic modules 151a to 151m may be provided to generate a set of insights (as discussed in relation to FIG. 3 below). The algorithmic modules 151 may include modules used to identify correlations between prompts and/or responses to prompts within a single decision tree or across two or more decision trees. The insights, in some aspects, may lead to updating at least one decision tree. For example, the algorithmic modules may identify that a particular behavior/response associated with a first prompt in a first decision related to a first area of human activity is correlated with a particular (and unexpected) behavior or response to a second prompt in a second decision tree related to a second area of human activity. In some aspects, the first and second decision tree are a same decision tree related to a same area of human activity, while in other aspects, the first and second decision trees are different decision trees related to different areas of human activity.

The algorithmic modules 151 and/or the machine learning modules 153 may also include algorithms or machine-trained modules for additional analyses. In some aspects, the analytics module 150 may be specific to an individual system operator and may initially include a configured set of ‘standard’ algorithms available to all users of the system. Analytics module 150 may include a first set of algorithmic modules for identifying correlations between prompts in different decision trees, a second set of algorithmic modules for identifying correlations between one or more responses to prompts for a first patient at a plurality of times, and/or a set of machine learning-based modules for optimizing a set of decision trees, e.g., updating the one or more prompts or the recommendations made (e.g., recommending a particular intervention or additional assessment to perform). The anonymized data, whether from one provider or aggregated from multiple providers, may be made available as a commoditized or public data set for researchers to perform queries or to generate insights. The users of the anonymized data may generate additional analysis algorithms (e.g., analysis programs or machine trained networks) based on novel analyses of the anonymized data to address different questions or issues. The user-generated algorithms may be stored in a centralized analytics module 150 and may be available for retrieval via a local software (or hardware) client associated with an individual provider. For example, a particular user or researcher may define an analysis to be performed on the anonymized data to generate insights and/or improve the decision trees and make the algorithm or the set of weights associated with nodes of a machine-trained network available to others.

FIG. 2 is a diagram 200 illustrating a data collection and anonymization operation for decision tree response data received at a plurality of local providers 225A-225K in accordance with aspects of the disclosure. Diagram 200 illustrates that each of a plurality of local providers may receive decision tree responses (e.g., one of 225A to 225K) associated with a plurality of decision trees (e.g., decision trees 220-1, 220-2, 220-3, and 220-N) for storage in a local response/patient data storage 230A to 230K. The local response/patient data storage 230K may include patient data 232 or patient data 233. Patient data 232 or 233 may include data related to a current state 232a or 233a and time-series data 232b or 233b that tracks the state variables over time. Each response/patient data storage 230A to 230K may provide the received data to a corresponding local anonymizer 235A to 235K before providing the anonymized data to the centralized anonymized data storage 240 in the cloud 245. The anonymized data storage 240 may include data structures (e.g., response set 247 and response set 248) identifying related sets of responses. Each response set (e.g., response set 247 and 248) may include an anonymized ID (e.g., anonymized ID 247a or anonymized ID 248a) that does not include patient-identifying information but that may be used within a HIPAA compliant system to identify a patient associated with the response set. Each response set (e.g., response set 247 and 248) may further include association data (e.g., association data 247b or association data 248b) regarding responses to prompts in a set of decision trees. For example, decision tree responses 225K and local response/patient data storage 230K may be associated with a set of components in a local system 215. By aggregating data from multiple providers administering the plurality of decision trees, the centralized anonymized data storage 240 may enable more meaningful and/or accurate analyses.

FIG. 3 is a diagram 300 illustrating the use of anonymized data in an anonymized data storage 340 to generate insights and update a set of decision trees stored in decision tree storage 365 in accordance with aspects of the disclosure. Diagram 300 illustrates that a set of decision tree responses 325 regarding a plurality of decision trees 320-1, 320-2, 320-3, and 320-N may be provided to an anonymized data storage 340. The anonymized data storage 340 may include data structures (e.g., response set 347 and response set 348) identifying related sets of responses. Each response set (e.g., response set 347 and 348) may include an anonymized ID (e.g., anonymized ID 347a or anonymized ID 348a) that does not include patient-identifying information but that may be used within a HIPAA compliant system to identity a patient associated with the response set. Each response set (e.g., response set 347 and 348) may further include association data (e.g., association data 347b or association data 348b) regarding responses to prompts in a set of decision trees. A set of algorithmic modules 351 including algorithmic modules 351 to 351m may retrieve or obtain data from the structured data stored in the anonymized data storage 340 (e.g., via one or more queries of the structured data) for one or more algorithmic analyses. The algorithmic analyses may include regressions or other algorithmic operations to identify a set of clinical insights 360 that may be used to update a set of decision trees in a decision tree storage 365. The decision tree storage 365, in some aspects, corresponds to the decision tree storage 120, while in other aspects, the decision tree storage 365 may be a centralized decision tree storage from which a set of local decision tree storages 120 may obtain decision trees (e.g., based on a push or pull operation between the local decision tree storage 120 and the centralized decision tree storage 365).

The clinical insights 360 may include identifying correlations between elements of the decision trees (e.g., decision trees 320-1 to 320-N). The identified correlations may indicate relationships between issues experienced by patients. Based on the identified correlations and related clinical insights 360 at least one element of at least one decision tree may be updated. For example, if the set of algorithmic modules 351 identifies a previously-unrecognized correlation between a first issue (e.g., a first dental issue) and a second issue (e.g., one or more of a second dental issue, a behavioral issue, a sleep issue, a feeding issue, or an issue associated with another decision tree) at least one element of one of the correlated decision trees may be updated. For example, if a particular behavioral issue is identified as being correlated with a particular dental issue, a recommended assessment and/or treatment element of at least one decision tree associated with the particular behavioral and/or dental issue may be updated to suggest an assessment and/or treatment related to the correlated dental issue and/or behavioral issue, respectively.

In other aspects, the update may relate to a connection or flow between prompt elements within a single decision tree. For example, a hierarchy of prompts (e.g., an ordered list of prompts that are presented based on responses to previous prompts) may be flattened (or dependencies between prompts in the ordered list of prompts may be changed) if a correlation between a response to a prompt at a higher level of the hierarchy is identified as not being correlated with a response to a prompt at a lower level of the hierarchy such that presenting the lower-level prompt should not depend on a response to the higher-level prompt. In some aspects, the clinical insight may indicate a lack of a relationship between issues experienced by patients that had been assumed in the structure of at least one decision tree. The update may then relate to decoupling the prompts or removing a particular assessment and/or treatment recommendation based on at least one response to a prompt related to one of the issues that were previously assumed to be connected and/or correlated.

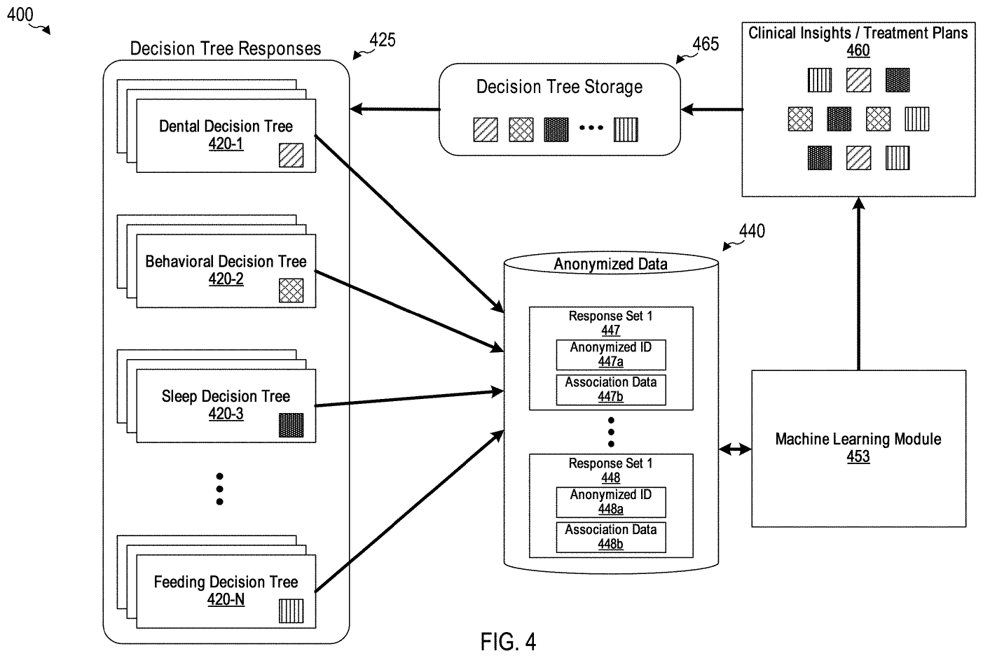

FIG. 4 is a diagram 400 illustrating the use of anonymized data in an anonymized data storage 440 to generate insights and update a set of decision trees stored in decision tree storage 465 in accordance with aspects of the disclosure. Diagram 400 illustrates that a set of decision tree responses 425 regarding a plurality of decision trees 420-1, 420-2, 420-3, and 420-N may be provided to an anonymized data storage 440. The anonymized data storage 440 may include data structures (e.g., response set 447 and response set 448) identifying related sets of responses. Each response set (e.g., response set 447 and 448) may include an anonymized ID (e.g., anonymized ID 447a or anonymized ID 448a) that does not include patient-identifying information but that may be used within a HIPAA compliant system to identify a patient associated with the response set. Each response set (e.g., response set 447 and 448) may further include association data (e.g., association data 447b or association data 448b) regarding responses to prompts in a set of decision trees. A machine learning module 453 may retrieve or obtain data from the structured data stored in the anonymized data storage 440 (e.g., via one or more queries of the structured data) for one or more machine-learning operations. The machine-learning operations, in some aspects, may include a first set of machine-training operations to train a machine-trained network to perform a second set of inference operations based on the machine-trained network. The machine-learning operations may be used to generate clinical insights and/or treatment plans. As shown, the machine-trained network in some aspects, may identify optimized treatment recommendations (e.g., clinical insights/treatment plans 460) that can be used to update a set of recommendations associated with one or more decision trees stored in decision tree storage 465. Alternatively, the machine-trained network may be used to perform an inference for a particular patient based on a set of state variables. For example, a machine-training operation performed by machine learning module 453 may identify, based on a training set of historical data from anonymized data storage 440 an optimized (and holistic) treatment plan based on a state associated with a particular patient. The decision tree storage 465, in some aspects, corresponds to the decision tree storage 120, while in other aspects, the decision tree storage 465 may be a centralized decision tree storage from which a set of local decision tree storages 120 may obtain decision trees (e.g., based on a push or pull operation between the local decision tree storage 120 and the centralized decision tree storage 465).

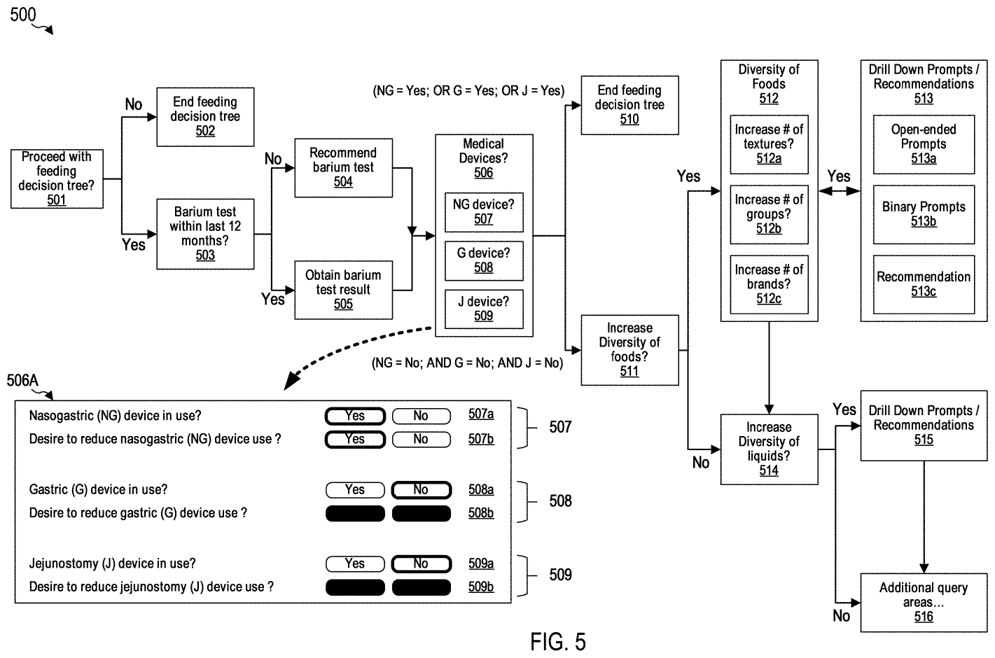

FIG. 5 is a diagram 500 illustrating a portion of a feeding decision tree, in accordance with some aspects of the disclosure. The feeding decision tree may begin by outputting a prompt 501 that asks whether the user would like to proceed with the feeding decision tree. If a “No” response is received, the system may end a feeding decision tree (e.g., by outputting a prompt 502 indicating that no further prompts. If, however, a “Yes” response, a prompt 503 asking whether a barium swallow test has been conducted within the last 12 months.

If a “No” response is received to prompt 503, a recommendation 504 to perform a barium swallow test may be presented. Presenting the recommendation 504 may include, in some aspects, presenting a selectable element to indicate that the user has received the recommendation and is ready to proceed to a next prompt. If a “Yes” response is received to prompt 503, a request 505 for the results of the barium swallow test may be presented/output to the user. After receiving the results of the barium swallow tests, or after receiving an indication that a user is ready to proceed to a next prompt, or set of prompts, 506 addressing medical devices relating to feeding. For example, a set of prompts 507, 508, and 509, may be presented to determine whether a nasogastric (NG) tube, a gastric (G) tube, or a jejunostomy (J) tube, respectively, is currently in use and/or whether a user desires to reduce a dependence on the corresponding device. The prompts 507-509, in some aspects, may be presented as an ordered set of prompts, while in other aspects, the prompt 506 addressing the medical devices may be implemented as a set of related sub-prompts as shown in prompt 506A. Prompt 506A, in some aspects, may include sets of prompts 507, 508, and 509 related to a NG tube, a G tube, or a J tube respectively. Each of the sets of prompts, 507, 508, and 509, in some aspects, may include a first sub-prompt, e.g., 507a, 508a, and 509a, as to the existence and/or use of the device of the particular device and a second sub-prompt, e.g., 507b, 508b, 509b that may be presented, or be made selectable, if a “yes” element is selected for the corresponding first sub-prompt.

Based on the response to one or more of the prompts in the set of prompts 506, the feeding decision tree may determine, and output, a next prompt. For example, if any of the devices are in use (e.g., (NG=Yes; OR G=Yes; OR J=Yes)) the feeding decision tree may determine to present a prompt indicating the end of the feeding decision tree. If, however, no feeding device is in use (e.g., ((NG=No; ANDG=No; AND J=No)), the feeding decision tree may determine to present a prompt 511 relating to whether there is a desire to increase the diversity of solid foods consumed by the patient. If a “yes” response to prompt 511 is received, the feeding decision tree may present a set of food diversity prompts 512 including a set of prompts relating to different aspects of food diversity. For example, the set of food diversity prompts 512 may include a first prompt 512a relating to whether there is a desire to increase a number of food textures consumed by the patient, a second prompt 512b relating to whether there is a desire to increase a number of food groups consumed by the patient, or a third prompt 512c relating to whether there is a desire to increase a number of brands consumed by the patient.

If a “yes” response is received to any of prompts 512a, 512b, or 512c, the feeding decision tree may present a set of additional (drill down) prompts and/or recommendations 513. For example, a drill down prompt may include one or more open-ended prompts 513a or one or more binary prompts 513b that may be used to identify a goal for the patient. An open-ended prompt in the one or more open-ended prompts 513a may provide a text-entry element for user input, while the binary prompts in the one or more binary prompts 513b may include one or more prompts regarding specific textures, food groups, and/or brands with binary input options (a set of radio buttons that can be toggled on or off, or a set of yes/no input elements, etc.). Based on the responses received to the previously presented prompts, the feeding decision tree may identify a recommendation in the set of recommendations 513c. The recommendations 513c may be presented as they are identified or may be stored for a summarized recommendation at the conclusion of the feeding decision tree.

If a “no” response is received to the prompt 511 or the set of food diversity prompts 512 and additional (drill down) prompts and/or recommendations 513 have been responded to, the feeding decision tree may present a prompt 514 relating to whether there is a desire to increase the diversity of liquids consumed by the patient. If a “yes” response to prompt 514 is received, the feeding decision tree may present a set of drill down prompts and/or recommendation 515. The set of drill down prompts and/or recommendation 515 may include a set of prompts similar to prompts 512a, 512b, and 512c except for relating to liquids instead of solid foods. Additional prompts similar to prompts 513a and/or 513b may be presented based on a first set of responses to a first set of prompts in the set of drill down prompts and/or recommendation 515. The feeding decision tree may identify a recommendation in a set of recommendations in the set of drill down prompts and/or recommendation 515. The feeding decision tree may continue through a set of prompts related to additional areas of interest 516. For example, the additional areas of interest may include throat, esophagus, and/or chewing concerns; mealtime behavior; and a level of concern regarding feeding issues.

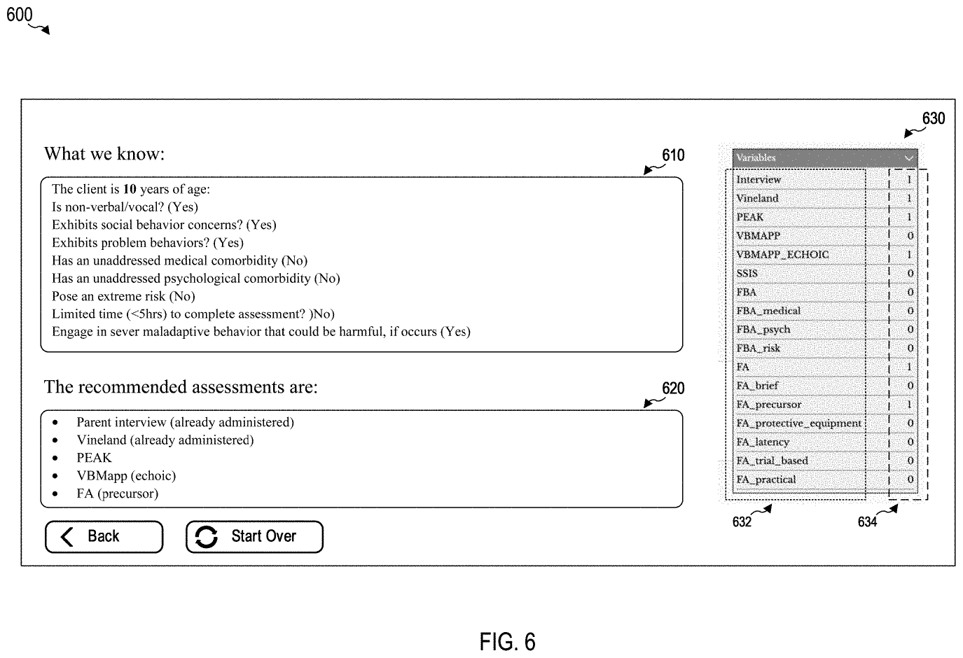

FIG. 6 is a diagram 600 illustrating a first example display that may be presented to a user resulting from traversing a decision tree in accordance with some aspects of the disclosure. Diagram 600 includes a summary area 610 for presenting a summary of the responses to prompts in a related decision tree. In some aspects, the summary may present a subset of the responses to the prompts in the related decision tree that may be relevant to a clinician. The diagram 600 may further include a recommendation area 620 that indicates a set of recommended actions (and whether they have already been implemented). The recommendations, in some aspects, may relate to one or more assessments and/or one or more recommended interventions. The recommendations, in some aspects, may be based on the set of responses to the prompts in the related tree. In some aspects, the recommendations may further be based on responses to prompts in previously traversed decision trees associated the same patient or user.

A state panel 630 may also be displayed along with the summary area 610 and the recommendation area 620. The state panel 630, in some aspects, identifies a set of relevant state variables 632 that may be used to define a state of a patient. A set of state values 634 may be associated with the set of relevant state variables 632 to indicate a state associated with a particular patient or user. The set of state values 634 for the set of relevant state variables 632 may be determined by traversing a set of decision trees. For example, a first variable may relate to an assessment associated with a first decision tree (e.g., a vineland variable may indicate whether a vineland assessment associated with a behavioral decision tree has been performed), while additional variables may relate to other assessments or other relevant variables associated with other decision trees (e.g., a PEAK variable may indicate whether a PEAK assessment associated with a language/speech decision tree has been performed). While the set of relevant state variables 632 is shown in diagram 600 to be associated with a binary set of state values 634, in other aspects, the set of state values 634 may include other formats such as values in a continuous range of values or an integer from a set of integers (e.g., an age of a patient may be recorded as an integer or as a non-integer value indicating portions of a year based on a birthdate and a current date.

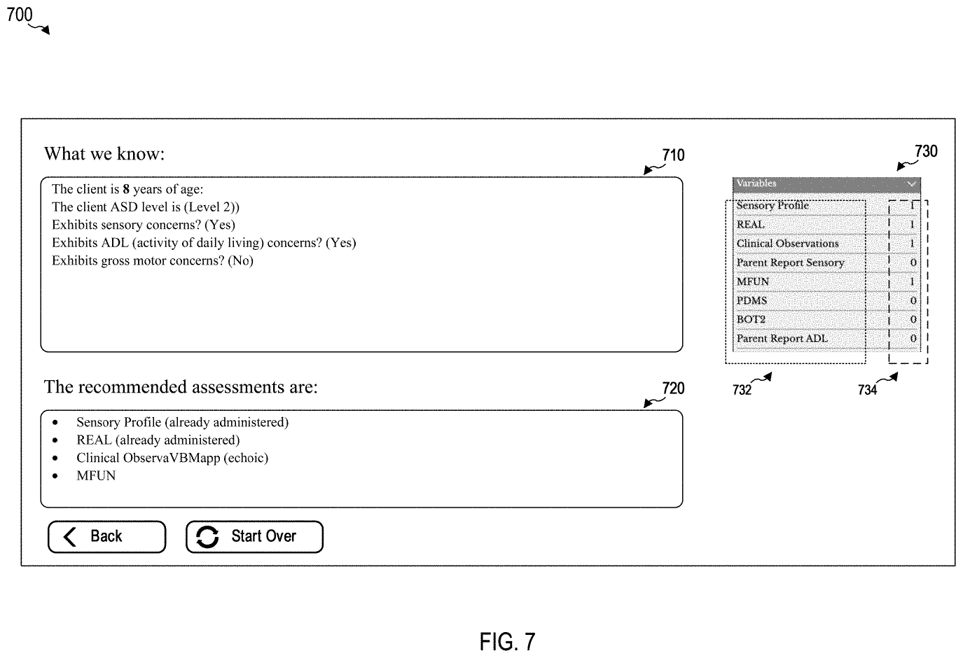

FIG. 7 is a diagram 700 illustrating a second example display that may be presented to a user resulting from traversing a second decision tree in accordance with some aspects of the disclosure. Diagram 700 includes a summary area 710 for presenting a summary of the responses to prompts in a related decision tree. In some aspects, the summary may present a subset of the responses to the prompts in the related decision tree that may be relevant to a clinician. The diagram 700 may further include a recommendation area 720 that indicates a set of recommended actions (and whether they have already been implemented). The recommendations, in some aspects, may relate to one or more assessments and/or one or more recommended interventions. The recommendations, in some aspects, may be based on the set of responses to the prompts in the related tree. In some aspects, the recommendations may further be based on responses to prompts in previously traversed decision trees associated the same patient or user.

Similarly to the state panel 630 of FIG. 6, a state panel 730 may also be displayed along with the summary area 710 and the recommendation area 720. The state panel 730, in some aspects, identifies a set of relevant state variables 732 that may be used to define a state of a patient. A set of state values 734 may be associated with the set of relevant state variables 732 to indicate a state associated with a particular patient or user. The set of state values 734 for the set of relevant state variables 732 may be determined by traversing a set of decision trees.

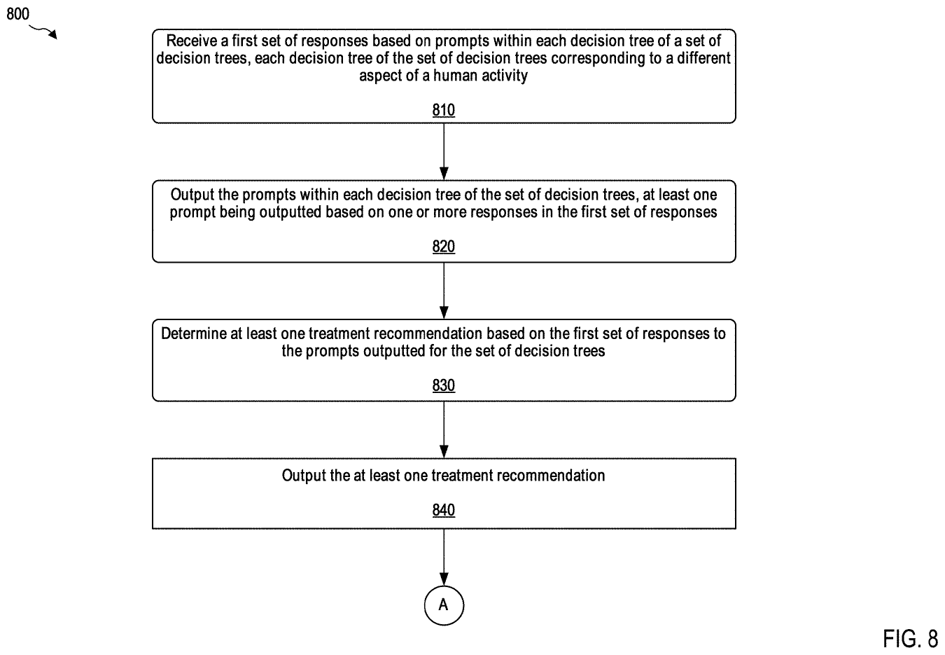

FIG. 8 is a flowchart 800 for a method of providing treatment recommendations based on a holistic assessment including a set of decision trees. The method may be performed by a system (e.g., system 100 or components of system 100 such as I/O interfaces 110, decision tree storage 120, and response/patient data storage 130). At 810, the system may receive (or obtain) a first set of responses based on prompts within each decision tree of a set of decision trees, each decision tree of the set of decision trees corresponding to a different aspect of a human activity. In some aspects, the set of decision trees may include at least one of (1) a dental decision tree relating to one or more of a set of dental issues, a set of dental assessments, or a set of dental interventions, (2) a behavioral decision tree relating to one or more of a set of behavioral issues, a set of behavioral assessments, or a set of behavioral interventions, (3) a sleep disorder decision tree relating to one or more of a set of sleep disorder issues, a set of sleep disorder assessments, or a set of sleep disorder interventions, and (4) a feeding decision tree relating to one or more of a set of nutritional issues, a set of nutritional assessments, or a set of nutritional interventions. The first set of responses, in some aspects, is related to a first patient being treated for an autism spectrum disorder and the at least one treatment recommendation is an autism treatment recommendation. For example, referring to FIGS. 1-5, the system 100 may receive (or obtain), via input interfaces in I/O interfaces 110, responses 225K, 325, 425 to a set of prompts (e.g., prompts 501, 503, 506-509, 511, 516) associated with one or more of decision trees 120-1 to 120-N, stored in decision tree storage 120.

In some aspects, receiving the first set of responses at 810 is part of a larger response collection operation. For example, in some aspects, receiving the first set of responses at 810 may further include receiving additional sets of responses related to a plurality of additional patients based on prompts within each decision tree of the set of decision trees. Alternatively, or additionally, receiving the first set of responses at 810, in some aspects, the system may also receive (or obtain) additional sets of responses at a plurality of different times related to the first patient based on prompts within each decision tree of the set of decision trees. In some aspects, the system may also receive (or obtain) an additional set of responses based on prompts within each decision tree of the set of decision trees for each of a plurality of additional patients being treated for an autism spectrum disorder.

At 820, the system, in some aspects, may output the prompts within each decision tree of the set of decision trees. In some aspects, at least one prompt may be outputted based on one or more responses in the first set of responses. Accordingly, operations associated with receiving the first set of responses at 810 and outputting the prompts at 820 may, in some aspects, be interleaved. For example, referring to FIGS. 1-5, the system 100 may output, via output interfaces in I/O interfaces 110, a set of prompts (e.g., prompts 501, 503, 506-509, 511, 516) in each decision tree (e.g., one or more of decision trees 120-1 to 120-N) stored in decision tree storage 120. Further, referring to FIG. 5, the prompts (e.g., alternative prompts 502 and 503; 504 and 505; 510 and 511; etc.) output within the feeding decision tree are dependent on the responses to other prompts (e.g., prompt 501, 503, 506-509, etc., respectively).

At 830, the system may determine at least one treatment recommendation based on the first set of responses to the prompts outputted for the set of decision trees. In some aspects, the at least one treatment recommendation includes at least one of an additional assessment to be performed or a therapeutic intervention to be implemented. In some aspects, the system may also output an indication of a set of responses upon which determining the at least one treatment recommendation at 830 is based. The indication of the set of responses, in some aspects, may include a state value associated with at least one variable associated with at least one decision tree in the set of decision trees. For example, referring to FIGS. 1 and 5-7, the system 100 may output the prompts 501-516 associated with the feeding decision tree 120-N, or prompts associated with different decision trees to determine a set of recommendations 504, 513c, 620 or 720 and the output (e.g., a user interface on a display device of system 100) may include an indication of a set of responses 610 or 710 and/or a set of relevant state variables 732 and a corresponding set of state values 734 defining a state of the patient or user that is used in determining the at least one treatment recommendation at 830.

Finally, at 840, they system may output the at least one treatment recommendation determined at 830. The at least one treatment recommendation may be one or more of an additional assessment to be performed or a therapeutic intervention to be implemented. In some aspects, the at least one treatment recommendation may include a list of suggested assessments or therapeutic interventions and an indication that a subset of the list has already been performed and/or implemented. For example, referring to FIGS. 1 and 5-7, the system 100 may, based on the received responses stored in response/patient data storage 130, output the recommendations 504 and/or 513c or the recommendation 620 and 720 as illustrated in diagrams 600 and 700.

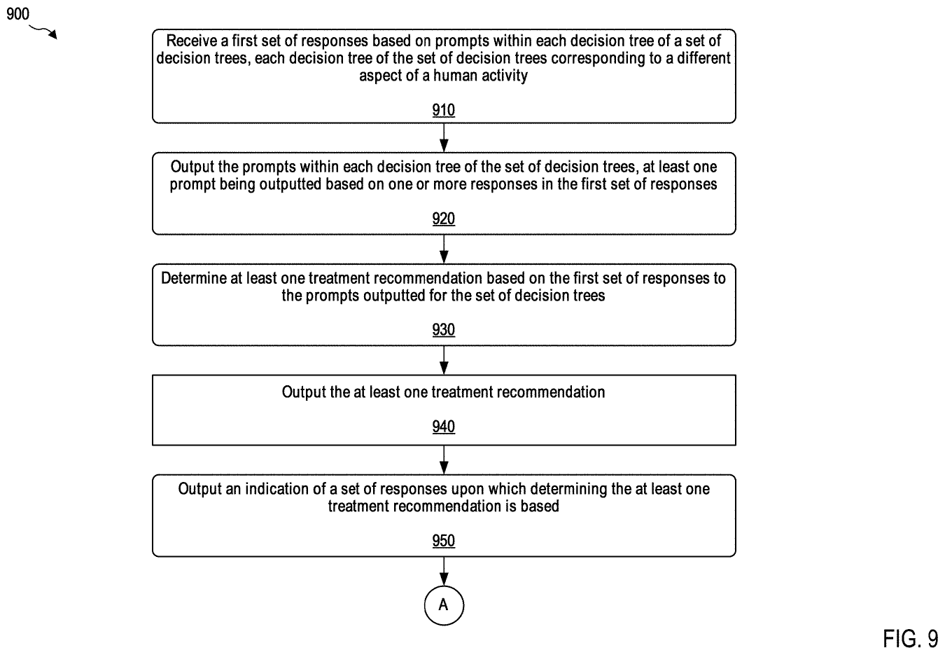

FIG. 9 is a flowchart 900 for a method of providing treatment recommendations based on a holistic assessment including a set of decision trees. The method may be performed by a system (e.g., system 100 or components of system 100 such as I/O interfaces 110, decision tree storage 120, and response/patient data storage 130). At 910, the system may receive (or obtain) a first set of responses based on prompts within each decision tree of a set of decision trees, each decision tree of the set of decision trees corresponding to a different aspect of a human activity. In some aspects, the set of decision trees may include at least one of (1) a dental decision tree relating to one or more of a set of dental issues, a set of dental assessments, or a set of dental interventions, (2) a behavioral decision tree relating to one or more of a set of behavioral issues, a set of behavioral assessments, or a set of behavioral interventions, (3) a sleep disorder decision tree relating to one or more of a set of sleep disorder issues, a set of sleep disorder assessments, or a set of sleep disorder interventions, and (4) a feeding decision tree relating to one or more of a set of nutritional issues, a set of nutritional assessments, or a set of nutritional interventions. The first set of responses, in some aspects, is related to a rust patient being treated for an autism spectrum disorder and the at least one treatment recommendation is an autism treatment recommendation. For example, referring to FIGS. 1-5, the system 100 may receive (or obtain), via input interfaces in I/O interfaces 110, responses 225K, 325, 425 to a set of prompts (e.g., prompts 501, 503, 506-509, 511, 516) associated with one or more of decision trees 120-1 to 120-N, stored in decision tree storage 120.

In some aspects, receiving the first set of responses at 910 is part of a larger response collection operation. For example, in some aspects, receiving the first set of responses at 910 may further include receiving additional sets of responses related to a plurality of additional patients based on prompts within each decision tree of the set of decision trees. Alternatively, or additionally, receiving the first set of responses at 910, in some aspects, the system may also receive (or obtain) additional sets of responses at a plurality of different times related to the first patient based on prompts within each decision tree of the set of decision trees. In some aspects, the system may also receive (or obtain) an additional set of responses based on prompts within each decision tree of the set of decision trees for each of a plurality of additional patients being treated for an autism spectrum disorder.

At 920, the system, in some aspects, may output the prompts within each decision tree of the set of decision trees. In some aspects, at least one prompt may be outputted based on one or more responses in the first set of responses. Accordingly, operations associated with receiving the first set of responses at 910 and outputting the prompts at 920 may, in some aspects, be interleaved. For example, referring to FIGS. 1-5, the system 100 may output, via output interfaces in I/O interfaces 110, a set of prompts (e.g., prompts 501, 503, 506-509, 511, 516) in each decision tree (e.g., one or more of decision trees 120-1 to 120-N) stored in decision tree storage 120. Further, referring to FIG. 5, the prompts (e.g., alternative prompts 502 and 503; 504 and 505; 510 and 511; etc.) output within the feeding decision tree are dependent on the responses to other prompts (e.g., prompt 501, 503, 506-509, etc., respectively).

At 930, the system may determine at least one treatment recommendation based on the first set of responses to the prompts outputted for the set of decision trees. In some aspects, the at least one treatment recommendation includes at least one of an additional assessment to be performed or a therapeutic intervention to be implemented. In some aspects, the system may also output an indication of a set of responses upon which determining the at least one treatment recommendation at 930 is based. The indication of the set of responses, in some aspects, may include a state value associated with at least one variable associated with at least one decision tree in the set of decision trees. For example, referring to FIGS. 1 and 5-7, the system 100 may output the prompts 501-516 associated with the feeding decision tree 120-N, or prompts associated with different decision trees to determine a set of recommendations 504, 513c, 620 or 720 and the output (e.g., a user interface on a display device of system 100) may include an indication of a set of responses 610 or 710 and/or a set of relevant state variables 732 and a corresponding set of state values 734 defining a state of the patient or user that is used in determining the at least one treatment recommendation at 930.

At 940, they system may output the at least one treatment recommendation determined at 930. The at least one treatment recommendation may be one or more of an additional assessment to be performed or a therapeutic intervention to be implemented. In some aspects, the at least one treatment recommendation may include a list of suggested assessments or therapeutic interventions and an indication that a subset of the list has already been performed and/or implemented. For example, referring to FIGS. 1 and 5-7, the system 100 may, based on the received responses stored in response/patient data storage 130, output the recommendations 504 and/or 513c or the recommendation 620 and 720 as illustrated in diagrams 600 and 700.

Finally, at 950, the system may output an indication of a set of responses upon which determining the at least one treatment recommendation is based. The indication of the set of responses, in some aspects, may include a state value associated with at least one variable associated with at least one decision tree in the set of decision trees. For example, referring to FIGS. 6 and 7, the user interface may include a summary area 610 or 710 indicating a set of responses and a state panel 630 or 730 indicating a set of relevant state variables 632 or 732 and a set of state values 634 or 734.

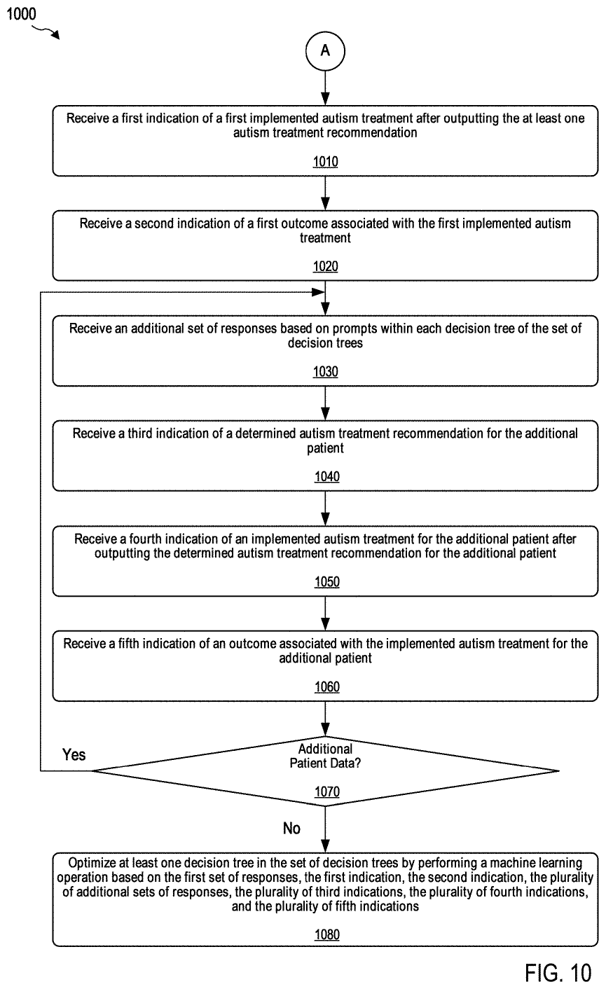

FIG. 10 is a flowchart 1000 for a method of optimizing at least one decision tree based on a set of responses in accordance with some aspects of the disclosure. The method illustrated in flowchart 1000, in some aspects, may be performed after the method illustrated in flowchart 800 or flowchart 900 as indicated by the letter “A” at the end of flowchart 800 and 900 and at the beginning of flowchart 1000. The method may be performed by a system (e.g., system 100 or components 140 and 150 of system 100). At 1010, the system (e.g., an anonymized data structure) may receive (or obtain) a first indication of a first implemented autism treatment after outputting the at least one autism treatment recommendation, e.g., at 840 or 940. The first indication may be received as part of an additional set of responses to a set of prompts output after the recommendation made at 840 or 940. For example, referring to FIGS. 1-4, the system 100 may receive (or obtain) an indication of an implemented autism treatment in a set of responses 225A-225K, 325, or 425 received via input interface of the I/O interfaces 110 that may be stored in a time-series data 132b, 133b, 1b, or 233b from one or more anonymizers (e.g., 135, 235A-235K, 335, or 435).

At 1020, the system (e.g., an anonymized data structure) may receive (or obtain) a second indication of a first outcome associated with the first implemented autism treatment. The second indication, in some aspects, may be associated with a value measuring a post-treatment state (or attribute) associated with the first patient. For example, the second indication may be received as part of an additional set of responses to a set of prompts output after the recommendation made at 840 or 940.

At 1030, the system (e.g., an anonymized data structure) may receive (or obtain), for each patient in a plurality of additional patients being treated for an autism spectrum disorder, an additional set of responses based on prompts within each decision tree of the set of decision trees. The additional sets of responses may include sets of responses that relate to different subsets of decision trees in the set of decision trees. Accordingly, the additional sets of responses may represent an exploration of a decision-tree/prompt/response space (e.g., a conceptual space defined by the possible decision trees, prompts, and responses). For example, referring to FIGS. 1-4, the system 100, or more specifically, the anonymized data storage 140, 240, 340, or 440 may receive (or obtain) response data (anonymized response data) based on decision tree responses 225A-225K, 325, or 425 from one or more anonymizers (e.g., 135, 235A-235K, 335, or 435).

At 1040, the system (e.g., an anonymized data structure) may receive (or obtain), for each patient in the plurality of additional patients, a third indication of a determined autism treatment recommendation for the patient in the plurality of additional patients. The third indication for a patient in the plurality of additional patients may indicate a set of one or more autism treatment recommendations and/or assessment recommendations. The set of one or more autism treatment recommendations and/or assessment recommendations, in some aspects, may include at least one of an additional assessment to be performed or a therapeutic intervention to be implemented similar to the at least one treatment recommendation determined at 830 or 930. For example, referring to FIGS. 1 and 5-7, the system 100 may output the prompts 501-516 associated with the feeding decision tree 120-N. or prompts associated with different decision trees to determine a set of recommendations 504, 513c, 620 or 720 for each patient in the plurality of additional patients and the output (e.g., a user interface on a display device of system 100) may include an indication of a set of responses 610 or 710 and/or a set of relevant state variables 732 and a corresponding set of state values 734 defining a state of the patient or user that is used in determining the at least one treatment recommendation at 1040.

At 1050, the system (e.g., an anonymized data structure) may receive (or obtain), for each patient in the plurality of additional patients, a fourth indication of a first implemented autism treatment after outputting at least one autism treatment recommendation for the patient in the plurality of additional patients (e.g., corresponding to outputting the autism recommendation for the first patient at 840 or 940). The third indication may be received as part of an additional set of responses to a set of prompts output after a recommendation for the patient in the plurality of additional patients corresponding to the recommendation made at 840 or 940 for the first patient. For example, referring to FIGS. 1-4, the system 100 may receive (or obtain) an indication of an implemented autism treatment for each of the plurality additional patients in a set of responses 225A-225K, 325, or 425 received via input interface of the I/O interfaces 110 that may be stored in a time series data 132b, 133b, 232b, or 233b from one or more anonymizers (e.g., 135, 235A-235K, 335, or 435).

At 1060, the system (e.g., an anonymized data structure) may receive (or obtain), for each patient in the plurality of additional patients, a fifth indication of an outcome associated with the implemented autism treatment for the patient. The fifth indication, in some aspects, may be associated with a value measuring a post-treatment state (or attribute) associated with one of the plurality of additional patients. For example, the second indication may be received as part of an additional set of responses to a set of prompts output after the recommendation made at 840 or 940.

At 1070, the system may determine if there is additional patient data to receive (or obtain). The determination may be based on determining whether all data in an anonymized data storage responsive to a query associated with flowchart 1000 has been processed. If the system determines that there is additional patient data to obtain for processing, the system may proceed to collect additional patient data at 1030 to 1070.

If the system determines, at 1070, that there is no additional patient data to receive (or obtain), the system may proceed to optimize, at 1080, at least one decision tree in the set of decision trees by performing a machine learning operation based on the first set of responses, the first indication, the second indication, the plurality of additional sets of responses, the plurality of third indications, the plurality of fourth indications, and the plurality of fifth indications. In some aspects, optimizing the at least one decision tree comprises updating at least one of (1) a prompt in the at least one decision tree, (2) the at least one prompt outputted based on the one or more responses in the first set of responses, or (3) at least one autism treatment recommendation based on a particular set of responses to a set of prompts within at least one decision tree. In some aspects, performing the machine learning operation includes performing a machine-learning-based operation to identify recommendations that optimize the value.

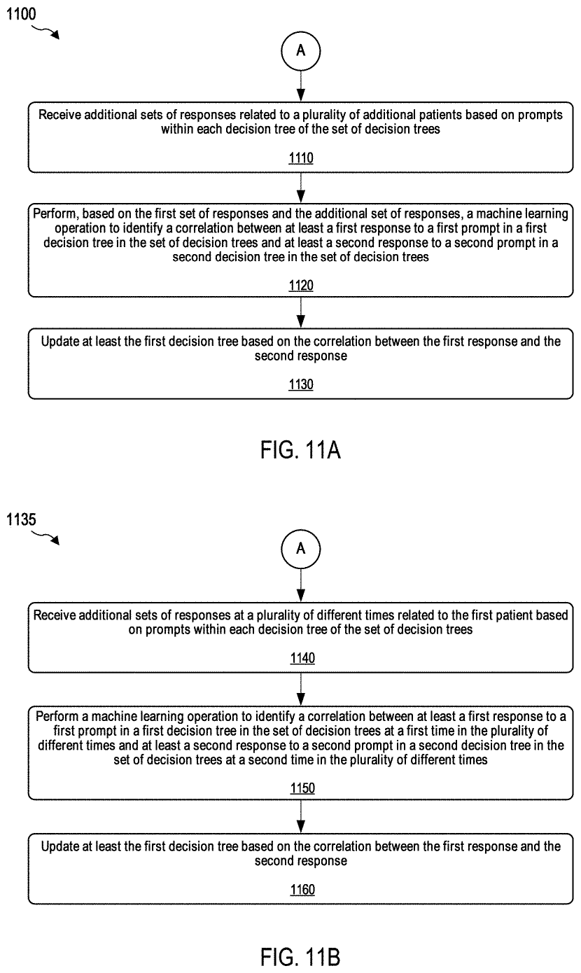

FIG. 11A is a flowchart 1100 for a method of updating at least a first decision tree based on a set of responses in accordance with some aspects of the disclosure. The method illustrated in flowchart 1100, in some aspects, may be performed after the method illustrated in flowchart 800 or flowchart 900 as indicated by the letter “A” at the end of flowchart 800 and 900 and at the beginning of flowchart 1100. The method may be performed by a system (e.g., system 100 or components 140 and 150 of system 100). At 1110, the system (e.g., an anonymized data structure) may receive (or obtain) additional sets of responses related to a plurality of additional patients based on prompts within each decision tree of the set of decision trees. The additional sets of responses may include sets of responses for different patients that relate to different subsets of decision trees in the set of decision trees. Accordingly, the additional sets of responses may represent an exploration of a decision-tree/prompt/response space (e.g., a conceptual space defined by the possible decision trees, prompts, and responses). For example, referring to FIGS. 1-4, the system 100, or more specifically, the anonymized data storage 140, 240, 340, or 440 may receive (or obtain) response data (anonymized response data) based on decision tree responses 225A-225K, 325, or 425 from one or more anonymizers (e.g., 135, 235A-235K, 335, or 435).

At 1120, the system, in some aspects, may perform, based on the first set of responses and the additional set of responses, a machine learning operation to identify a correlation between at least a first response to a first prompt in a first decision tree in the set of decision trees and at least a second response to a second prompt in a second decision tree in the set of decision trees. The machine learning operation, in some aspects, may be a regression-based analysis for identifying and/or quantifying correlations. For example, referring to FIGS. 3 and 4, algorithmic modules 351 (including algorithmic module 351a to algorithmic module 351m) and/or machine learning module 453 may be used to generate, based on data stored in anonymized data storage 340 or 440, clinical insights 360 or clinical insights/treatment plans 460 relating to correlations between elements of at least two decision trees. The correlations identified by the application of the algorithmic modules 351 or machine learning module 453 may indicate connections between elements of the at least two decision trees that were not previously appreciated (e.g., were not intuitive) and that may indicate an additional recommendation for assessing a patient, or for an intervention, based on receiving a particular response to one or both of the identified connected elements.

Finally, at 1130, the system, in some aspects, may update at least the first decision tree based on the correlation between the first response and the second response. In some aspects, updating that at least the first decision tree at 1130 may include updating a recommendation associated with the first response to recommend addressing the second prompt in the second decision tree. For example, referring to FIGS. 1, 3, and 4, the decision tree storage 120, 365, or 465 may updated based on analysis performed by analytics module 150 including algorithmic modules 151 and or machine learning module 153 used to generate clinical insights 360 or clinical insights/treatment plans 460.

FIG. 11B is a flowchart 1135 for a method of updating at least a first decision tree based on a set of responses in accordance with some aspects of the disclosure. The method illustrated in flowchart 1135, in some aspects, may be performed after the method illustrated in flowchart 800 or flowchart 900 as indicated by the letter “A” at the end of flowchart 800 and 900 and at the beginning of flowchart 1135. The method may be performed by a system (e.g., system 100 or components 140 and 150 of system 100). At 1140, the system (e.g., a response/patient data storage and/or an anonymized data structure) may receive (or obtain) additional sets of responses at a plurality of different times related to the first patient based on prompts within each decision tree of the set of decision trees. The additional sets of responses may include sets of responses for the patient related that relate to different subsets of decision trees in the set of decision trees as the patient changes over time (e.g., as issues arise or are addressed). Accordingly, the additional sets of responses may represent the progress of the patient over time. For example, referring to FIGS. 1-4, the system 100, or more specifically, the anonymized data storage 140, 240, 340, or 440 may receive (or obtain) response data (e.g., with personally-identifying information, or anonymized response data) associated with a non-personally identifying patient/user identifier (based on a set of decision tree data decision tree responses 225A-225K, 325, or 425 from one or more anonymizers (e.g., 135, 235A-235K, 335, or 435).

At 1150, the system, in some aspects, may perform, a machine learning operation to identify a correlation between at least a first response to a first prompt in a first decision tree in the set of decision trees at a first time in the plurality of different times and at least a second response to a second prompt in a second decision tree in the set of decision trees at a second time in the plurality of different times. The machine learning operation, in some aspects, may be a regression-based analysis for identifying and/or quantifying correlations. For example, referring to FIGS. 1, 3, and 4, algorithmic modules 351 and/or machine learning module 453 may be used to generate, based on data stored in anonymized data storage 340 or 440, clinical insights 360 or clinical insights/treatment plans 460 relating to correlations between elements of at least two decision trees at a same time or across time. The correlations identified by the application of the algorithmic modules 351 or machine learning module 453 may indicate connections between elements of at least two decision trees at a same time or across time that were not previously appreciated (e.g., were not intuitive) and that may indicate an additional recommendation for assessing a patient, or for an intervention, based on receiving a particular response to one or both of the identified connected elements.

Finally, at 1160, the system, in some aspects, may update at least the first decision tree based on the correlation between the first response and the second response. In some aspects, updating that at least the first decision tree at 1130 may include updating a recommendation associated with the first response to recommend addressing the second prompt in the second decision tree. For example, referring to FIGS. 1, 3, and 4, the decision tree storage 120, 365, or 465 may updated based on analysis performed by analytics module 150 including algorithmic modules 151 and or machine learning module 153 used to generate clinical insights 360 or clinical insights/treatment plans 460.

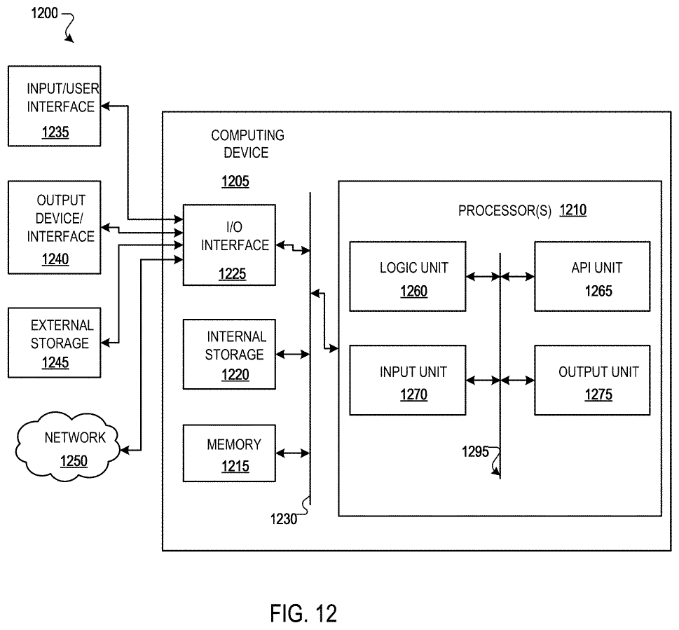

FIG. 12 illustrates an example computing environment with an example computer device suitable for use in some example implementations. Computer device 1205 in computing environment 1200 can include one or more processing units, cores, or processors 1210, memory 1215 (e.g., RAM, ROM, and/or the like), internal storage 1220 (e.g., magnetic, optical, solid-state storage, and/or organic), and/or IO interface 1225, any of which can be coupled on a communication mechanism or bus 1230 for communicating information or embedded in the computer device 1205. IO interface 1225 is also configured to receive images from cameras or provide images to projectors or displays, depending on the desired implementation.

Computer device 1205 can be communicatively coupled to input/user interface 1235 and output device/interface 1240. Either one or both of the input/user interface 1235 and output device/interface 1240 can be a wired or wireless interface and can be detachable. Input/user interface 1235 may include any device, component, sensor, or interface, physical or virtual, that can be used to provide input (e.g., buttons, touch-screen interface, keyboard, a pointing/cursor control, microphone, camera, braille, motion sensor, accelerometer, optical reader, and/or the like). Output device/interface 1240 may include a display, television, monitor, printer, speaker, braille, or the like. In some example implementations, input/user interface 1235 and output device/interface 1240 can be embedded with or physically coupled to the computer device 1205. In other example implementations, other computer devices may function as or provide the functions of input/user interface 1235 and output device/interface 1240 for a computer device 1205.

Examples of computer device 1205 may include, but are not limited to, highly mobile devices (e.g., smartphones, devices in vehicles and other machines, devices carried by humans and animals, and the like), mobile devices (e.g., tablets, notebooks, laptops, personal computers, portable televisions, radios, and the like), and devices not designed for mobility (e.g., desktop computers, other computers, information kiosks, televisions with one or more processors embedded therein and/or coupled thereto, radios, and the like).

Computer device 1205 can be communicatively coupled (e.g., via IO interface 1225) to external storage 1245 and network 1250 for communicating with any number of networked components, devices, and systems, including one or more computer devices of the same or different configuration. Computer device 1205 or any connected computer device can be functioning as, providing services of, or referred to as a server, client, thin server, general machine, special-purpose machine, or another label.

IO interface 1225 can include but is not limited to, wired and/or wireless interfaces using any communication or IO protocols or standards (e.g., Ethernet, 1202.11x, Universal system Bus, WiMax, modem, a cellular network protocol, and the like) for communicating information to and/or from at least all the connected components, devices, and network in computing environment 1200. Network 1250 can be any network or combination of networks (e.g., the Internet, local area network, wide area network, a telephonic network, a cellular network, satellite network, and the like).

Computer device 1205 can use and/or communicate using computer-usable or computer readable media, including transitory media and non-transitory media. Transitory media include transmission media (e.g., metal cables, fiber optics), signals, carrier waves, and the like. Non-transitory media include magnetic media (e.g., disks and tapes), optical media (e.g., CI) ROM, digital video disks, Blu-ray disks), solid-state media (e.g., RAM, ROM, flash memory, solid-state storage), and other non-volatile storage or memory.

Computer device 1205 can be used to implement techniques, methods, applications, processes, or computer-executable instructions in some example computing environments. Computer-executable instructions can be retrieved from transitory media, and stored on and retrieved from non-transitory media. The executable instructions can originate from one or more of any programming, scripting, and machine languages (e.g., C, C++, C#, Java, Visual Basic, Python, Perl, JavaScript, and others).

Processor(s) 1210 can execute under any operating system (OS) (not shown), in a native or virtual environment. One or more applications can be deployed that include logic unit 1260, application programming interface (API) unit 1265, input unit 1270, output unit 1275, and inter-unit communication mechanism 1295 for the different units to communicate with each other, with the OS, and with other applications (not shown). The described units and elements can be varied in design, function, configuration, or implementation and are not limited to the descriptions provided. Processor(s) 1210 can be in the form of hardware processors such as central processing units (CPUs) or in a combination of hardware and software units.

In some example implementations, when information or an execution instruction is received by API unit 1265, it may be communicated to one or more other units (e.g., logic unit 1260, input unit 1270, output unit 1275). In some instances, logic unit 1260 may be configured to control the information flow among the units and direct the services provided by API unit 1265, the input unit 1270, the output unit 1275, in some example implementations described above. For example, the flow of one or more processes or implementations may be controlled by logic unit 1260 alone or in conjunction with API unit 1265. The input unit 1270 may be configured to obtain input for the calculations described in the example implementations, and the output unit 1275 may be configured to provide an output based on the calculations described in example implementations.

Processor(s) 1210 can be configured receive a first set of responses based on prompts within each decision tree of a set of decision trees, each decision tree of the set of decision trees corresponding to a different aspect of a human activity. The processor(s) 1210 may also be configured to output the prompts within each decision tree of the set of decision trees, at least one prompt being outputted based on one or more responses in the first set of responses. The processor(s) 1210 may further be configured to determine at least one treatment recommendation based on the first set of responses to the prompts outputted for the set of decision trees. The processor(s) 1210 may further be configured to output the at least one treatment recommendation. The processor(s) 1210 may also be configured to output an indication of a set of responses upon which determining the at least one treatment recommendation is based. The processor(s) 1210 may also be configured to receive a first indication of a first implemented autism treatment after outputting the at least one autism treatment recommendation. The processor(s) 1210 may also be configured to receive a second indication of a first outcome associated with the first implemented autism treatment. The processor(s) 1210 may further be configured to receiving for each of a plurality of additional patients being treated for an autism spectrum disorder: an additional set of responses based on prompts within each decision tree of the set of decision trees; a third indication of a determined autism treatment recommendation for the additional patient; a fourth indication of an implemented autism treatment for the additional patient after outputting the determined autism treatment recommendation for the additional patient; and a fifth indication of an outcome associated with the implemented autism treatment for the additional patient. The processor(s) 1210 may further be configured to optimize at least one decision tree in the set of decision trees by performing a machine learning operation based on the first set of responses, the first indication, the second indication, the plurality of additional sets of responses, the plurality of third indications, the plurality of fourth indications, and the plurality of fifth indications. The processor(s) 1210 may further be configured to receive additional sets of responses related to a plurality of additional patients based on prompts within each decision tree of the set of decision trees. The processor(s) 1210 may further be configured to perform, based on the first set of responses and the additional set of responses, a machine learning operation to identify a correlation between at least a first response to a first prompt in a first decision tree in the set of decision trees and at least a second response to a second prompt in a second decision tree in the set of decision trees. The processor(s) 1210 may further be configured to update at least the first decision tree based on the correlation between the first response and the second response. The processor(s) 1210 may further be configured to receive additional sets of responses at a plurality of different times related to the first patient based on prompts within each decision tree of the set of decision trees. The processor(s) 1210 may further be configured to perform a machine learning operation to identify a correlation between at least a first response to a first prompt in a first decision tree in the set of decision trees at a first time in the plurality of different times and at least a second response to a second prompt in a second decision tree in the set of decision trees at a second time in the plurality of different times. The processor(s) 1210 may further be configured to updating at least the first decision tree based on the correlation between the first response and the second response.

Some portions of the detailed description are presented in terms of algorithms and symbolic representations of operations within a computer. These algorithmic descriptions and symbolic representations are the means used by those skilled in the data processing arts to convey the essence of their innovations to others skilled in the art. An algorithm is a series of defined steps leading to a desired end state or result. In example implementations, the steps carried out require physical manipulations of tangible quantities for achieving a tangible result.

Unless specifically stated otherwise, as apparent from the discussion, it is appreciated that throughout the description, discussions utilizing terms such as “processing,” “computing.” “calculating,” “determining,” “displaying,” or the like, can include the actions and processes of a computer system or other information processing device that manipulates and transforms data represented as physical (electronic) quantities within the computer system's registers and memories into other data similarly represented as physical quantities within the computer system's memories or registers or other information storage, transmission or display devices.

Example implementations may also relate to an apparatus for performing the operations herein. This apparatus may be specially constructed for the required purposes, or it may include one or more general-purpose computers selectively activated or reconfigured by one or more computer programs. Such computer programs may be stored in a computer readable medium, such as a computer readable storage medium or a computer readable signal medium. A computer readable storage medium may involve tangible mediums such as, but not limited to optical disks, magnetic disks, read-only memories, random access memories, solid-state devices, and drives, or any other types of tangible or non-transitory media suitable for storing electronic information. A computer readable signal medium may include mediums such as carrier waves. The algorithms and displays presented herein are not inherently related to any particular computer or other apparatus. Computer programs can involve pure software implementations that involve instructions that perform the operations of the desired implementation.

Various general-purpose systems may be used with programs and modules in accordance with the examples herein, or it may prove convenient to construct a more specialized apparatus to perform desired method steps. In addition, the example implementations are not described with reference to any particular programming language. It will be appreciated that a variety of programming languages may be used to implement the teachings of the example implementations as described herein. The instructions of the programming language(s) may be executed by one or more processing devices, e.g., central processing units (CPUs), processors, or controllers.

As is known in the art, the operations described above can be performed by hardware, software, or some combination of software and hardware. Various aspects of the example implementations may be implemented using circuits and logic devices (hardware), while other aspects may be implemented using instructions stored on a machine-readable medium (software), which if executed by a processor, would cause the processor to perform a method to carry out implementations of the present application. Further, some example implementations of the present application may be performed solely in hardware, whereas other example implementations may be performed solely in software. Moreover, the various functions described can be performed in a single unit, or can be spread across a number of components in any number of ways. When performed by software, the methods may be executed by a processor, such as a general-purpose computer, based on instructions stored on a computer readable medium. If desired, the instructions can be stored on the medium in a compressed and/or encrypted format.

Moreover, other implementations of the present application will be apparent to those skilled in the art from consideration of the specification and practice of the teachings of the present application. Various aspects and/or components of the described example implementations may be used singly or in any combination. It is intended that the specification and example implementations be considered as examples only, with the true scope and spirit of the present application being indicated by the following claims.

What is claimed:

1. A method, comprising:
receiving a first set of responses based on prompts within each decision tree of a set of decision trees, each decision tree of the set of decision trees corresponding to a different aspect of a human activity;
outputting the prompts within each decision tree of the set of decision trees, at least one prompt being outputted based on one or more responses in the first set of responses;
determining at least one treatment recommendation based on the first set of responses to the prompts outputted for the set of decision trees; and
outputting the at least one treatment recommendation.

2. The method of claim 1, wherein the set of decision trees comprises at least one of (1) a dental decision tree relating to one or more of a set of dental issues, a set of dental assessments, or a set of dental interventions, (2) a behavioral decision tree relating to one or more of a set of behavioral issues, a set of behavioral assessments, or a set of behavioral interventions, (3) a sleep disorder decision tree relating to one or more of a set of sleep disorder issues, a set of sleep disorder assessments, or a set of sleep disorder interventions, or (4) a feeding decision tree relating to one or more of a set of nutritional issues, a set of nutritional assessments, or a set of nutritional interventions.

3. The method of claim 2, wherein the set of decision trees comprises a plurality of decision trees.

4. The method of claim 1, wherein the at least one treatment recommendation comprises at least one of an additional assessment to be performed or a therapeutic intervention to be implemented.

5. The method of claim 1, further comprising:
outputting an indication of a set of responses upon which determining the at least one treatment recommendation is based.

6. The method of claim 5, wherein the indication of the set of responses includes a state value associated with at least one variable associated with at least one decision tree in the set of decision trees.

7. The method of claim 1, wherein the first set of responses is related to a first patient being treated for an autism spectrum disorder and the at least one treatment recommendation is at least one autism treatment recommendation.

8. The method of claim 7, further comprising:
receiving a first indication of a first implemented autism treatment after outputting the at least one autism treatment recommendation; and
receiving a second indication of a first outcome associated with the first implemented autism treatment.

9. The method of claim 8, further comprising:
receiving for each of a plurality of additional patients being treated for an autism spectrum disorder:
an additional set of responses based on prompts within each decision tree of the set of decision trees;
a third indication of a determined autism treatment recommendation for the patient in the plurality of additional patients;
a fourth indication of an implemented autism treatment for the patient in the plurality of additional patients after outputting the determined autism treatment recommendation for the patient in the plurality of additional patients; and
a fifth indication of an outcome associated with the implemented autism treatment for the patient in the plurality of additional patients; and
optimizing at least one decision tree in the set of decision trees by performing a machine learning operation based on the first set of responses, the first indication, the second indication, the sets of responses for the plurality of additional patients, the third indications for the plurality of additional patients, the fourth indications for the plurality of additional patients, and the fifth indications for the plurality of additional patients.

10. The method of claim 9, wherein optimizing the at least one decision tree comprises updating at least one of (1) a prompt in the at least one decision tree, (2) the at least one prompt outputted based on the one or more responses in the first set of responses, or (3) at least one autism treatment recommendation based on a particular set of responses to a set of prompts within at least one decision tree.

11. The method of claim 9, wherein each of the second indication and the filth indication are associated with a value measuring a post-treatment state (or attribute) associated with one of the first patient or one of the plurality of additional patients, and performing the machine learning operation comprises performing a machine-learning-based operation to identify recommendations that optimize the value.

12. The method of claim 1, wherein the first set of responses is related to a first patient, the method further comprising:
receiving additional sets of responses related to a plurality of additional patients based on prompts within each decision tree of the set of decision trees;
performing, based on the first set of responses and the additional set of responses, a machine learning operation to identify a correlation between at least a first response to a first prompt in a first decision tree in the set of decision trees and at least a second response to a second prompt in a second decision tree in the set of decision trees; and
updating at least the first decision tree based on the correlation between the first response and the second response.

13. The method of claim 12, wherein updating at least the first decision tree comprises updating a recommendation associated with the first response to recommend addressing the second prompt in the second decision tree.

14. The method of claim 1, wherein the first set of responses is related to a first patient being treated for an autism spectrum disorder and the at least one treatment recommendation is an autism treatment recommendation, the method further comprising:
receiving additional sets of responses at a plurality of different times related to the first patient based on prompts within each decision tree of the set of decision trees;
performing a machine learning operation to identify a correlation between at least a first response to a first prompt in a first decision tree in the set of decision trees at a first time in the plurality of different times and at least a second response to a second prompt in a second decision tree in the set of decision trees at a second time in the plurality of different times; and
updating at least the first decision tree based on the correlation between the first response and the second response.

15. The method of claim 14, wherein the first time is the second time.

16. An apparatus comprising:
a memory storing a program for providing treatment recommendations based on a holistic assessment including a set of decision trees; and
at least one processor coupled to the memory that when executing the program, is configured to:
receive a first set of responses based on prompts within each decision tree of a set of decision trees, each decision tree of the set of decision trees corresponding to a different aspect of a human activity;
output the prompts within each decision tree of the set of decision trees, at least one prompt being outputted based on one or more responses in the first set of responses;
determine at least one treatment recommendation based on the first set of responses to the prompts outputted for the set of decision trees; and
output the at least one treatment recommendation.

17. The apparatus of claim 16, the at least one processor further being configured to:
output an indication of a set of responses upon which determining the at least one treatment recommendation is based.

18. The apparatus of claim 16, wherein the first set of responses is related to a first patient being treated for an autism spectrum disorder and the at least one treatment recommendation is at least one autism treatment recommendation, the at least one processor further configured to:
receive a first indication of a first implemented autism treatment after outputting the at least one autism treatment recommendation;
receive a second indication of a first outcome associated with the first implemented autism treatment;
receive for each patient in a plurality of additional patients being treated for an autism spectrum disorder:
an additional set of responses based on prompts within each decision tree of the set of decision trees for the patient in the plurality of additional patients;
a third indication of a determined autism treatment recommendation for the patient in the plurality of additional patients;
a fourth indication of an implemented autism treatment for the patient in the plurality of additional patients after outputting the determined autism treatment recommendation for the patient in the plurality of additional patients; and
a fifth indication of an outcome associated with the implemented autism treatment for the patient in the plurality of additional patients; and
optimize at least one decision tree in the set of decision trees by performing a machine learning operation based on the first set of responses, the first indication, the second indication, the sets of responses for the plurality of additional patients, the third indications for the plurality of additional patients, the fourth indications for the plurality of additional patients, and the fifth indications for the plurality of additional patients.

19. The apparatus of claim 16, wherein the first set of responses is related to a first patient, the at least one processor further configured to:
receive additional sets of responses related to a plurality of additional patients based on prompts within each decision tree of the set of decision trees;
perform, based on the first set of responses and the additional set of responses, a machine learning operation to identity a correlation between at least a first response to a first prompt in a first decision tree in the set of decision trees and at least a second response to a second prompt in a second decision tree in the set of decision trees; and
update at least the first decision tree based on the correlation between the first response and the second response.

20. The apparatus of claim 16, wherein the first set of responses is related to a first patient being treated for an autism spectrum disorder and the at least one treatment recommendation is an autism treatment recommendation, the at least one processor further configured to:
receive additional sets of responses at a plurality of different times related to the first patient based on prompts within each decision tree of the set of decision trees;
perform a machine learning operation to identify a correlation between at least a first response to a first prompt in a first decision tree in the set of decision trees at a first time in the plurality of different times and at least a second response to a second prompt in a second decision tree in the set of decision trees at a second time in the plurality of different times; and
update at least the first decision tree based on the correlation between the first response and the second response.
